SEQUENCE LISTING (10 sequences)

SEQ ID NO: 1 - protein, 122 aa, Artificial Sequence
  Amino Acid sequence identified using molecular biology techniques.
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 2 - protein, 107 aa, Artificial Sequence
  Amino Acid sequence identified using molecular biology techniques.
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 3 - protein, 5 aa, Artificial Sequence
  Amino Acid sequence identified using molecular biology techniques.
DYAMH

SEQ ID NO: 4 - protein, 17 aa, Artificial Sequence
  Amino Acid sequence identified using molecular biology techniques.
TISWNSGSIGYADSVKG

SEQ ID NO: 5 - protein, 13 aa, Artificial Sequence
  Amino Acid sequence identified using molecular biology techniques.
DIQYGNYYYGMDV

SEQ ID NO: 6 - protein, 11 aa, Artificial Sequence
  Amino Acid sequence identified using molecular biology techniques.
RASQSVSSYLA

SEQ ID NO: 7 - protein, 7 aa, Artificial Sequence
  Amino Acid sequence identified using molecular biology techniques.
DASNRAT

SEQ ID NO: 8 - protein, 9 aa, Artificial Sequence
  Amino Acid sequence identified using molecular biology techniques.
QQRSNWPIT

SEQ ID NO: 9 - protein, 17 aa, Artificial Sequence
  Amino Acid sequence identified using molecular biology techniques.
DYYGAGSFYDGLYGMDV

SEQ ID NO: 10 - protein, 81 aa, Artificial Sequence
  Amino Acid sequence identified using molecular biology techniques.
DYAMHWVRQAPGKGLEWVSTISWNSGSIGYADSVKGRFTISRDNAKKSLYLQMNSLRAEDTALYYCAKDIQYGNYYYGMDV

(19) United States
(12) Patent Application Publication
(10) Pub. No.: US 2013/0315901 A1
(43) Pub. Date: Nov. 28, 2013

(54) NOVEL USES

(71) Applicants: Frederick J. Derosier, Research Triangle Park,, NC (US); Immanuel Freedman, King of Prussia, PA (US); Ole Graff, Research Triangle Park, NC (US); Richard A. Grove, Uxbridge (GB)

(72) Inventors: Frederick J. Derosier, Research Triangle Park,, NC (US); Immanuel Freedman, King of Prussia, PA (US); Ole Graff, Research Triangle Park, NC (US); Richard A. Grove, Uxbridge (GB)

(73) Assignee: Glaxo Group Limited, Greenford, Middlesex (GB)

(21) Appl. No.: 13/978,723

(22) PCT Filed: Jan. 10, 2012
(86) PCT No.: PCT/US12/20729

§ 371 Date: Jul. 9, 2013

Related U.S. Application Data
(60) Provisional application No. 61/431,194, filed on Jan. 10, 2011.

(51) Int. Cl. A61K 39/395

(52) U.S. Cl. CPC A61K 39/3955
USPC 424/133.1; 424/172.1; 424/142.1

(57) ABSTRACT

A method of treating, arresting or preventing a disease responsive to treatment with an anti-CD20 antibody in a patient suffering therefrom, comprising administering to the patient at least one sub-depleting dose of antiCD20 antibody is disclosed.

### FIELD OF INVENTION

The present invention relates to a dosing regimen for anti-CD20 antibodies to treat various diseases.

### BACKGROUND OF THE INVENTION

Multiple sclerosis (MS) is an inflammatory demyelinating disease of the central nervous system (CNS) which results in CNS lesions that may be physically associated with activated T cells and myelin-laden macrophages while B-lymphocytes (B cells) may populate lesion cores and perivascular spaces. Multiple sclerosis as defined herein includes clinically isolated syndromes (CIS) and clinical multiple sclerosis together with atypical demyelinating disease variants. Clinically isolated syndromes refer to disease conditions that may progress to clinical multiple sclerosis, including isolated optic neuritis, myelitis and brainstem syndrome. Clinical multiple sclerosis includes Relapsing Remitting Multiple Sclerosis (RRMS), Primary Progressive Multiple Sclerosis (PPMS) and Secondary Progressive Multiple Sclerosis (SPMS). Atypical demyelinating disease variants include Marburg variant disease and tumefactive multiple sclerosis, together with severe monophasic disorders including complete transverse myelitis and neuromyelitis optica (NMO).

Direct evidence that B cells contribute to the pathophysiology of multiple sclerosis comes from a study that showed that rituximab markedly reduced disease activity relative to placebo in patients with RRMS [Hauser, 2008]. Rituximab is a chimeric monoclonal antibody (comprised of human and mouse components) that selectively depletes B cells bearing cluster of differentiation 20 (CD20⁺ B cells). Humanized or fully human monoclonal antibodies may have more favorable pharmacokinetics, better efficacy and lower immunogenicity than chimeric antibodies.

Ofatumumab (OFA) is a fully human anti-CD20 monoclonal antibody. GlaxoSmithKline/Genmab studied ofatumumab in a placebo-controlled trial using an intravenous (IV) formulation (GEN414) [Sorensen, 2010]. Like rituximab, IV ofatumumab resulted in a significant reduction in brain lesions over a 24 week period.

Progressive multifocal leukoencephalopathy (PML) is a serious viral disease characterised by multiple and progressive damage or inflammation to the brain. PML is believed to be caused by a polyomavirus, named the JC virus (JCV) after the first patient in whom it was observed. It is believed that a significant proportion of the population is seropositive for antibodies to the JCV, but PML is generally only seen in patients who have a deficiency of their immune system. In particular, PML has been seen in MS patients being treated with immunosuppressive medications, such as natalizumab (Tysabri™) and the anti-CD20 antibody, Rituximab (Rituxan/MabThera™). It would be highly desirable to minimise the risk of PML and other opportunistic infections in the course of treating patients with B-cell depleting agents such as anti-CD20 antibodies.

### SUMMARY OF INVENTION

GlaxoSmithKline study OFA110867 established that the duration of peripheral B cell depletion increases with dose of ofatumumab[ARZERRA], an anti-CD20 antibody.

Thus, therapeutic efficacy with anti-CD20 antibody therapy could be maintained and controlled, whilst also reducing the incidence of infections (including JC virus variants) that may trigger relapse by opening the blood brain barrier or by activating pathogenic immune cells expressing Toll-Like Receptor 9 (TLR9), if partial B-cell depletion is achieved (i.e. by delivering “a sub-depleting dose”). Depleting the CD20⁺ B-cell subset may reduce the supply of mature B-cells (including pathogenic B-cells) for migration across the blood brain barrier, clonal expansion, and differentiation into plasma or memory cells.

Accordingly, the present invention provides a method of treating, arresting or preventing a disease responsive to treatment with an anti-CD20 antibody in a patient suffering therefrom, comprising administering to the patient at least one sub-depleting dose of anti-CD20 antibody.

The method may comprise the administration to the patient of a plurality of sub-depleting doses. In an embodiment, the sub-depleting dose is between about 0.3 mg and 100 mg of anti-CD20 antibody. In an embodiment, the sub-depleting dose is between about 3 mg and 60 mg of anti-CD20 antibody. In a particular embodiment, the sub-depleting dose is selected from 3 mg, 30 mg or 60 mg of anti-CD20 antibody.

The method may also comprise the administration to the patient of a tolerizing dose, wherein the tolerizing dose is administered to the patient prior to the delivery of the at least one sub-depleting dose. In a particular embodiment, the tolerizing dose is administered about 1 week prior to the delivery of the (first) at least one sub-depleting dose. In an embodiment, the tolerizing dose is between about 0.3 mg and 3 mg of anti-CD20 antibody. In a particular embodiment, the tolerizing dose is about 3 mg of anti-CD20 antibody.

In one embodiment, where multiple sub-depleting doses of anti-CD20 antibody are administered to the patient, the time elapsed between administrations of sub-depleting doses of anti-CD20 antibody is the lesser of the time to replete peripheral CD19⁺ B-cells to Lower Limit of Normal (about 110 cells/μL) or three month (approximately 12 week) intervals.

In an embodiment, the disease responsive to treatment with an anti-CD20 antibody is multiple sclerosis or rheumatoid arthritis.

In an embodiment, the anti-CD20 antibody is ofatumumab, orcrelizumab or rituximab. In a particular embodiment, the anti-CD20 antibody is ofatumumab.

In an embodiment, administration is by the subcutaneous (SC) route.

In one aspect, the present invention relates to a method for treating, arresting or preventing multiple sclerosis (including relapse-remitting multiple sclerosis, primary progressive multiple sclerosis or secondary progressive multiple sclerosis) or spino-optical sclerosis or neuromyelitis optica in a human patient comprising administering an anti-CD20 antibody at

(a) initial 3 mg dose followed by 30 mg at week one, and 30 mg at week 12; or
(b) intial 3 mg dose followed by 60 mg at week one, and 60 mg at week 12; or
(c) initial 3 mg dose followed by 60 mg at every four weeks for 24 weeks; or
(d) initial 3 mg dose followed by 30 mg at every four weeks for 24 weeks; or
(e) initial 3 mg dose, followed by 10 mg at every twelve weeks for 24 weeks.

The method may further comprise one or more additional or subsequent courses of therapy, comprising repeating the administration defined by (a), (b), (c), (d) or (e) above, optionally without the initial 3 mg dose.

In another aspect, the present invention relates treating, arresting or preventing multiple sclerosis (including relapse-remitting multiple sclerosis, primary progressive multiple sclerosis or secondary progressive multiple sclerosis) or spino-optical sclerosis or neuromyelitis optica in a human patient comprising administering an anti-CD20 antibody at

(a) 30 mg at week one, and 30 mg at week 12; or
(b) 60 mg at week one, and 60 mg at week 12; or
(c) 60 mg at every four weeks for 24 weeks; or
(d) 30 mg at every four weeks for 24 weeks; or
(e) 3 mg at week one, and 3 mg at week 12; or
(f) 10 mg at week one, and at every twelve weeks for 24 weeks.

In another aspect, the invention provides a method of treating, arresting or preventing rheumatoid arthritis (RA), comprising administering at least one dose of an anti-CD20 antibody at about 0.3 mg to about 100 mg, subcutaneously to the patient.

In another aspect, the invention provides a method of treating, arresting or preventing rheumatoid arthritis (RA), comprising administering an anti-CD20 antibody at

(a) initial 3 mg dose followed by 30 mg at week one, and 30 mg at week 12; or
(b) intial 3 mg dose followed by 60 mg at week one, and 60 mg at week 12; or
(c) initial 3 mg dose followed by 60 mg at every four weeks for 24 weeks; or
(d) initial 3 mg dose followed by 30 mg at every four weeks for 24 weeks; or
(e) initial 3 mg dose, followed by 10 mg at every twelve weeks for 24 weeks.

In another aspect, the invention provides a method of treating, arresting or preventing rheumatoid arthritis (RA), comprising administering an anti-CD20 antibody at

(a) 30 mg at week one, and 30 mg at week 12; or
(b) 60 mg at week one, and 60 mg at week 12; or
(c) 60 mg at every four weeks for 24 weeks; or
(d) 30 mg at every four weeks for 24 weeks; or
(e) 3 mg at week one, and 3 mg at week 12; or
(f) 10 mg at week one, and at every twelve weeks for 24 weeks.

The invention also provides an anti-CD20 antibody for the treatment of rheumatoid arthritis or multiple sclerosis (including relapse-remitting multiple sclerosis, primary progressive multiple sclerosis or secondary progressive multiple sclerosis) or spino-optical sclerosis or neuromyelitis optica in a human patient by the administration of said anti-CD20 antibody at

(a) 30 mg at week one, and 30 mg at week 12; or
(b) 60 mg at week one, and 60 mg at week 12; or
(c) 60 mg at every four weeks for 24 weeks; or
(d) 30 mg at every four weeks for 24 weeks; or
(e) 10 mg at every twelve weeks for 24 weeks.

The administration may under (a) to (e) may optionally comprise an initial dose of 3 mg on day 1.

### DETAILED DESCRIPTION

Dose Rationale

In one embodiment, dose predictions are based on data from a Rheumatoid Arthritis (RA) single SC dose study (GlaxoSmithKline OFA110867). The enhanced pharmacometric model includes compartments describing the time course of ofatumumab within a subcutaneous depot and the time course of pathogenic effector memory T- or memory B-cells selectively recruited across the blood brain barrier via adhesion. The T-cells may be antigen-primed CD4+ cells, possibly of CD45RO CCR7⁻ phenotype with mean proliferation and disappearance rates according to Table 2 of Macallan et al. [Macallan, 2004]. In principle, the T cells may alternatively be antigen-primed CD8⁺ cells. The uptake rate constants for pathogenic T- or B-cell migration across the blood brain barrier were based on their respective in vitro migration rates in the presence of both TNF-α and IFN-γ cytokines reported by Alter et al [Alter, 2003]. The pathogenic memory B-cells are considered to turnover in Cerebrospinal Fluid (CSF) at the rate estimated for peripheral CD19⁺ B-cells in study OFA110867 and to be cleared by anti-CD20 antibody by at least the ADCC pathway at a rate corresponding to in vitro studies including those reported by Bleeker et al [Bleeker, 2007]. The model assumes that pathogenic memory B-cells may not proliferate unless a specific antigen or non-specific infection triggers proliferation via antigen-binding or TLR9 pathways. The model further assumes that the instantaneous mean incidence rate of Gd-enhancing lesions may be substantially proportional to the count of peripherally-activated pathogenic T or B-cells present in CSF.

Although the 3, 30, and 60 mg single SC doses were all well-tolerated in study OFA110867, the 3 mg single dose was the most tolerable. The single SC 3 mg initial dose is intended to reduce cytokine release reaction to subsequent doses by depleting peripheral CD20⁺ B-cells by about 50% over about 6 to about 9 days. The 3 mg initial dose (Week 0), followed by the 60 mg mg monthly dose was chosen as the lowest monthly dose at which at least about 95% peripheral CD20⁺B-cell depletion is likely to be maintained substantially continuously in at least about 90% of the subjects. This dose is expected to be well-tolerated in view of minimal further cytokine release activity following the 3 mg initial dose.

Following the initial 3 mg SC dose, the 60 mg and 30 mg arms are expected to deplete peripheral B-cells to reduce activation of pathogenic T- and B-cells (via reduced antigen presentation or regulatory T-cell augmentation) to levels similar to those observed for the efficacious dose in Genmab study GEN 414 in the 100 mg arm (in which two 100 mg doses were administered two weeks apart). The time to replete peripheral CD19⁺ B-cells to Lower Limit of Normal (about 110 cells/μL) is predicted to exceed about 20 weeks after the last dose for doses greater than about 3 mg SC. If these doses are repeated at the lesser of time to replete B-cells to peripheral LLN or three month intervals, partial repletion of peripheral B-cells may provide some level of adaptive immunity against infections (including JC virus variants) that may trigger relapse by opening the blood brain barrier or by activating pathogenic immune cells expressing TLR9.

Kuenz et al. reported that CSF B-cells correlated with early brain inflammation in multiple sclerosis including number of Gd-enhancing lesions, while Petereit et al observed that reduction in lesion number correlated with reduction in CSF B-cell count [Kuenz, 2008; Petereit, 2008].

Depleting the CD20⁺ B-cell subset may reduce the supply of mature B-cells (including pathogenic B-cells) for migration across the blood brain barrier, clonal expansion, and differentiation into plasma or memory cells. Kuenz et al. reported that “new focal white-matter lesions appear to develop following new waves of inflammation, involving immune cells which enter the brain from the peripheral blood and cause major blood brain barrier leakage mediated by matrix metalloproteinases (MMP)” [Kuenz, 2008]. Sormani et al. reported that the statistical distribution of new Gd-enhancing lesions observed during a 24 week period was well described by a negative binomial distribution with expected value μ=13.0 and over-dispersion parameter Θ=0.52 in RRMS patients with monthly MRI scanning [Sormani, 2001]. These observations support the notion that immune cells are selectively recruited across the blood brain barrier in almost discrete events with an average inter-event interval of about 22 weeks, according to a Gamma-Poisson mixture model. The Sormani et al. model provides a statistical link for the mean rate of incidence of new Gd-enhancing lesions and pharmacometric model predictions of the reduction in count of peripherally-activated pathogenic B or T-cells that have migrated into CSF. Petereit and Rubbert-Roth showed that the observed rituximab plasma:CSF concentration for subjects with a normal blood brain barrier is about 0.1%, which is also expected for ofatumumab [Petereit, 2009]. Normal healthy serum:CSF ratios are about 230:1 for albumin and 369:1 for IgG in the absence of intrathecal IgG synthesis [Tourtellotte, 1975]. Hence for subjects with moderate to severe impairment of the blood brain barrier corresponding to an albumin index of 14-30 [Cook, 2006], the ofatumumab plasma:CSF concentration ratio is predicted to be in the range of about 0.87% to about 1.87%, based on the assumption that plasma:CSF ratios are reasonably close to serum:CSF ratios.

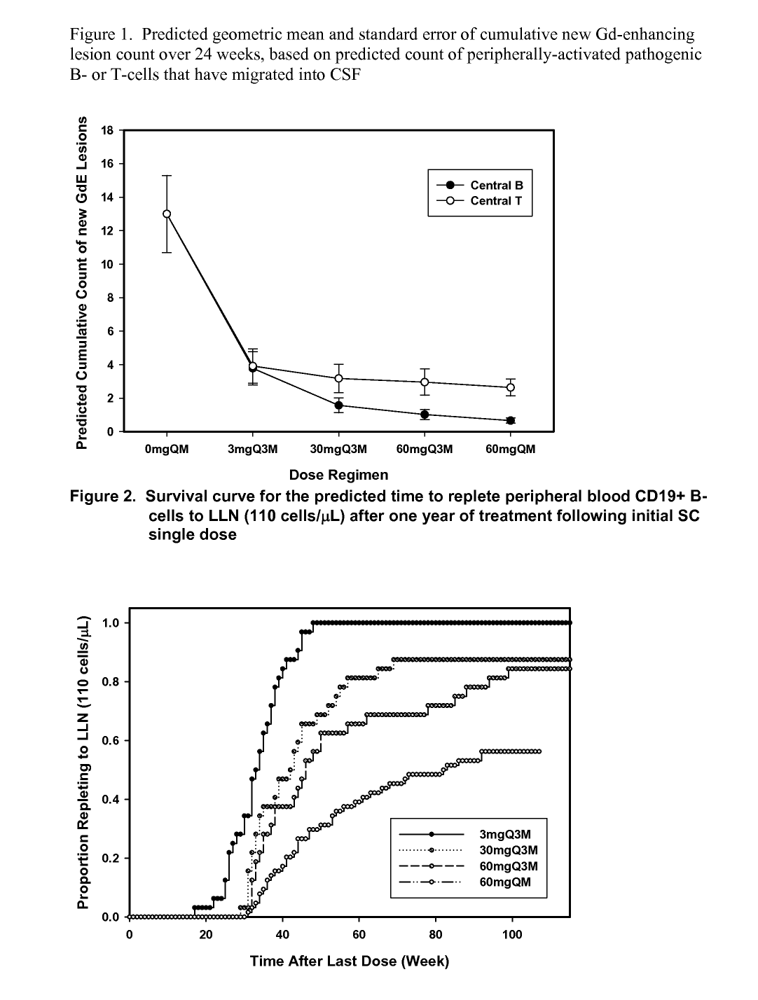

For the 60 mg monthly cohort, the expected rate of peripherally-activated T- and B-cell migration across the blood brain barrier is very small based on the predicted extensive peripheral B-cell depletion. Although moderate B-cell depletion in CSF (and perhaps CNS) may be possible via antibody-dependent-cell-mediated cytoxicity (ADCC) in accordance with the sigmoid Emax model of Bleeker et al., it is not expected to be robust or complete [Bleeker, 2007]. FIG. 1 shows the predicted geometric mean and standard error of new Gd-enhancing lesions based on the reduction of pathogenic T- and B-cells present in CSF that have been selectively recruited across the blood brain barrier via adhesion and the negative binomial distribution for unselected RRMS patients reported by Sormani et al. [Sormani, 2001].

FIG. 2 shows the survival curve for the predicted time to replete peripheral blood CD19⁺ B-cells to 110 cells/μL after dosing RRMS subjects with SC ofatumumab 3 mg every 3 months, 60 mg every 3 months, or 60 mg monthly for one year of treatment following an initial 3 mg SC dose. The initial distribution of peripheral CD19+ B-cells is drawn from a lognormal distribution with expected value μ=198 GI/L and standard deviation 0.403 on the logarithmic scale obtained by a maximum likelihood fit to the baseline peripheral CD19+ counts observed for the 100 mg cohort of study GEN 414 in RRMS patients.

In one embodiment of the invention, the anti-CD20 antibody is monoclonal.

In one embodiment, the anti-CD20 antibody has Fc mediated effector function.

In one embodiment, the anti-CD20 antibody has antibody-dependent-cell-mediated cytoxicity (ADCC) effector function.

In one embodiment, the anti-CD20 antibody has complement-dependent-cytoxicity (CDC) effector function.

In one embodiment of the invention, the anti-CD20 antibody is a chimeric, humanized or human monoclonal antibody.

In one embodiment, the monoclonal antibody against CD20 (anti-CD20 antibody) is a full-length antibody selected from the group consisting of a full-length IgG1 antibody, a full-length IgG2 antibody, a full-length IgG3 antibody, a full-length IgG4 antibody, a full-length IgM antibody, a full-length IgA1 antibody, a full-length IgA2 antibody, a full-length secretory IgA antibody, a full-length IgD antibody, and a full-length IgE antibody, wherein the antibody is glycosylated in a eukaryotic cell.

In one embodiment, the anti-CD20 antibody is a full-length antibody, such as a full-length IgG1 antibody.

In one embodiment, the anti-CD20 antibody is an antibody fragment, such as a scFv or a UniBody™ (a monovalent antibody as disclosed in WO 2007/059782).

In one embodiment of the invention, the antibody against CD20 (anti-CD20 antibody) is a binding-domain immunoglobulin fusion protein comprising (i) a binding domain polypeptide in the form of a heavy chain variable region of SEQ ID NO:1 or a light chain variable region of SEQ ID NO:2 that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region.

In one embodiment, the antibody against CD20 binds to mutant P172S CD20 (proline at position 172 mutated to serine) with at least the same affinity as to human CD20.

In one embodiment of the invention, the antibody against CD20 binds to an epitope on CD20
(i) which does not comprise or require the amino acid residue proline at position 172;
(ii) which does not comprise or require the amino acid residues alanine at position 170 or proline at position 172;
(iii) which comprises or requires the amino acid residues asparagine at position 163 and asparagine at position 166;
(iv) which does not comprise or require the amino acid residue proline at position 172, but which comprises or requires the amino acid residues asparagine at position 163 and asparagine at position 166; or
(v) which does not comprise or require the amino acid residues alanine at position 170 or proline at position 172, but which comprises or requires the amino acid residues asparagine at position 163 and asparagine at position 166.

In one embodiment, the antibody against CD20 binds to an epitope in the small first extracellular loop of human CD20.

In one embodiment, the antibody against CD20 binds to a discontinuous epitope on CD20.

In one embodiment, the antibody against CD20 binds to a discontinuous epitope on CD20, wherein the epitope comprises part of the first small extracellular loop and part of the second extracellular loop.

In one embodiment, the antibody against CD20 binds to a discontinuous epitope on CD20, wherein the epitope has residues AGIYAP of the small first extracellular loop and residues MESLNFIRAHTPYI of the second extracellular loop.

In one embodiment, the antibody against CD20 has one or more of the characteristics selected from the group consisting of:
(i) capable of inducing complement dependent cytotoxicity (CDC) of cells expressing CD20 in the presence of complement;
(ii) capable of inducing complement dependent cytotoxicity (CDC) of cells expressing CD20 and high levels of CD55 and/or CD59 in the presence of complement;
(iii) capable of inducing apoptosis of cells expressing CD20;
(iv) capable of inducing antibody dependent cellular cytotoxicity (ADCC) of cells expressing CD20 in the presence of effector cells;
(v) capable of inducing homotypic adhesion of cells which express CD20;
(vi) capable of translocating into lipid rafts upon binding to CD20;
(vii) capable of depleting cells expressing CD20;
(viii) capable of depleting cells expressing low levels of CD20 (CD20low cells); and
(ix) capable of effectively depleting B cells in situ in human tissues.

In one embodiment of the invention, the antibody against CD20 comprises a VH CDR3 sequence selected from SEQ ID NOs: 5, 9, or 11.

In one embodiment, the antibody against CD20 comprises a VH CDR1 of SEQ ID NO:3, a VH CDR2 of SEQ ID NO:4, a VH CDR3 of SEQ ID NO:5, a VL CDR1 of SEQ ID NO:6, a VL CDR2 of SEQ ID NO:7 and a VL CDR3 sequence of SEQ ID NO:8.

In one embodiment of the invention, the antibody against CD20 comprises a VH CDR1-CDR3 spanning sequence of SEQ ID NO: 10.

In one embodiment of the invention, the antibody against CD20 has human heavy chain and human light chain variable regions comprising the amino acid sequences as set forth in SEQ ID NO:1 and SEQ ID NO:2, respectively; or amino acid sequences which are at least 95% identical, and more preferably at least 98%, or at least 99% identical to the amino acid sequences as set forth in SEQ ID NO: 1 and SEQ ID NO:2, respectively.

In one embodiment of the invention an anti-CD20 antibody is selected from one of the anti-CD20 antibodies disclosed in WO 2004/035607, such as ofatumumab, 2F2, 11B8, or 7D8, one of the antibodies disclosed in WO 2005/103081, such as 2C6, one of the antibodies disclosed in WO 2004/103404, AME-133 (humanized and optimized anti-CD20 monoclonal antibody, developed by Applied Molecular Evolution), one of the antibodies disclosed in US 2003/0118592, TRU-015 (CytoxB20G, a small modular immunopharmaceutical fusion protein derived from key domains on an anti-CD20 antibody, developed by Trubion Pharmaceuticals Inc), one of the antibodies disclosed in WO 2003/68821, IMMU-106 (a humanized anti-CD20 monoclonal antibody), one of the antibodies disclosed in WO 2004/56312, ocrelizumab (2H7.v16, PRO-70769, R-1594), Bexxar® (tositumomab), and Rituxan®/MabThera® (rituximab). The terms “CD20” and “CD20 antigen” are used interchangeably herein, and include any variants, isoforms and species homologs of human CD20, which are naturally expressed by cells or are expressed on cells transfected with the CD20 gene. Synonyms of CD20, as recognized in the art, include B-lymphocyte surface antigen B1, Leu-16 and Bp35. Human CD20 has UniProtKB/Swiss-Prot entry P11836.

The term “immunoglobulin” as used herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH) and a heavy chain constant region. The heavy chain constant region, CH, typically is comprised of three domains, CH1, CH2, and CH3. Each light chain typically is comprised of a light chain variable region (abbreviated herein as VL) and a light chain constant region. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases, such as variable domain residue numbering as in Kabat or according to Kabat herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (for instance residue 52a according to Kabat) after residue 52 of VH CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “antibody” as used herein refers to an immunoglobulin molecule, a fragment of an immunoglobulin molecule, or a derivative of either thereof, which has the ability to specifically bind to an antigen under typical physiological conditions for a significant period of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or a time sufficient for the antibody to recruit an Fc-mediated effector activity).

The variable regions of the heavy and light chains of the immunoglobulin molecule contain a binding domain that interacts with an antigen. The constant regions of the antibodies may mediate the binding of the immunoglobulin to host tissues or factors, including various cells of the immune system (such as effector cells) and components of the complement system such as C1q, the first component in the classical pathway of complement activation.

The anti-CD20 antibody may be mono-, bi- or multispecific. Indeed, bispecific antibodies, diabodies, and the like, provided by the present invention may bind any suitable target in addition to a portion of CD20.

As indicated above, the term “antibody” as used herein, unless otherwise stated or clearly contradicted by the context, includes fragments of an antibody provided by any known technique, such as enzymatic cleavage, peptide synthesis and recombinant techniques that retain the ability to specifically bind to an antigen. It has been shown that the antigen-binding function of an antibody may be performed by fragments of a full-length (intact) antibody. Examples of antigen-binding fragments encompassed within the term “antibody” include, but are not limited to (i) a Fab fragment, a monovalent fragment consisting of the VL, VH, CL and CH1 domains; (ii) F(ab)2 and F(ab′)2 fragments, bivalent fragments comprising two Fab fragments linked by a disulfide bridge at the hinge region; (iii) a Fd fragment consisting essentially of the VH and CH 1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al. (November 2003) Trends Biotechnol. 21(11):484-90); (vi) a camelid antibody or nanobody (Revets et al. (January 2005) Expert Opin Biol Ther. 5(1):111-24), (vii) an isolated complementarity determining region (CDR), such as a VH CDR3, (viii) a UniBody™, a monovalent antibody as disclosed in WO 2007/059782, (ix) a single chain antibody or single chain Fv (scFv), see for instance Bird et al., Science 242, 423-426 (1988) and Huston et al., PNAS USA 85, 5879-5883 (1988)), (x) a diabody (a scFv dimer), which can be monospecific or bispecific (see for instance PNAS USA 90(14), 6444-6448 (1993), EP 404097 or WO 93/11161 for a description of diabodies), a triabody or a tetrabody. Although such fragments are generally included within the definition of an antibody, they collectively and each independently are unique features of the present invention, exhibiting different biological properties and utility. These and other useful antibody fragments in the context of the present invention are discussed further herein.

It should be understood that the term antibody generally includes monoclonal antibodies as well as polyclonal antibodies. The antibodies can be human, humanized, chimeric, murine, etc. An antibody as generated can possess any isotype.

The term “human antibody”, as used herein, is intended to include antibodies having variable and constant regions derived from human germline immunoglobulin sequences. The human antibodies of the present invention may include amino acid residues not encoded by human germline immunoglobulin sequences (for instance mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted into human framework sequences.

As used herein, a human antibody is “derived from” a particular germline sequence if the antibody is obtained from a system using human immunoglobulin sequences, for instance by immunizing a transgenic mouse carrying human immunoglobulin genes or by screening a human immunoglobulin gene library, and wherein the selected human antibody is at least 90%, such as at least 95%, for instance at least 96%, such as at least 97%, for instance at least 98%, or such as at least 99% identical in amino acid sequence to the amino acid sequence encoded by the germline immunoglobulin gene. Typically, a human antibody derived from a particular human germline sequence will display no more than 10 amino acid differences, such as no more than 5, for instance no more than 4, 3, 2, or 1 amino acid difference from the amino acid sequence encoded by the germline immunoglobulin gene. For VH antibody sequences the VH CDR3 domain is not included in such comparison.

The term “chimeric antibody” refers to an antibody that contains one or more regions from one antibody and one or more regions from one or more other antibodies. The term “chimeric antibody” includes monovalent, divalent, or polyvalent antibodies. A monovalent chimeric antibody is a dimer (HL)) formed by a chimeric H chain associated through disulfide bridges with a chimeric L chain. A divalent chimeric antibody is a tetramer (H2L2) formed by two HL dimers associated through at least one disulfide bridge. A polyvalent chimeric antibody may also be produced, for example, by employing a CH region that assembles into a molecule with 2+ binding sites (for instance from an IgM H chain, or μ chain). Typically, a chimeric antibody refers to an antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see for instance U.S. Pat. No. 4,816,567 and Morrison et al., PNAS USA 81, 6851-6855 (1984)). Chimeric antibodies are produced by recombinant processes well known in the art (see for instance Cabilly et al., PNAS USA 81, 3273-3277 (1984), Morrison et al., PNAS USA 81, 6851-6855 (1984), Boulianne et al., Nature 312, 643-646 (1984), EP125023, Neuberger et al., Nature 314, 268-270 (1985), EP171496, EP173494, WO 86/01533, EP184187, Sahagan et al., J. Immunol. 137, 1066-1074 (1986), WO 87/02671, Liu et al., PNAS USA 84, 3439-3443 (1987), Sun et al., PNAS USA 84, 214-218 (1987), Better et al., Science 240, 1041-1043 (1988) and Harlow et al., Antibodies: A Laboratory Manual, Cold Spring Harbor Laboratory Press, Cold Spring Harbor, N.Y., (1988)).

The term “humanized antibody” refers to a human antibody which contain minimal sequences derived from a non-human antibody. Typically, humanized antibodies are human immunoglobulins (recipient antibody) in which residues from a hypervariable region of the recipient are replaced by residues from a hypervariable region of a non-human species (donor antibody), such as mouse, rat, rabbit or non-human primate having the desired specificity, affinity, and capacity.

Furthermore, humanized antibodies may comprise residues which are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. A humanized antibody optionally also will comprise at least a portion of a human immunoglobulin constant region. For further details, see Jones et al., Nature 321, 522-525 (1986), Riechmann et al., Nature 332, 323-329 (1988) and Presta, Curr. Op. Struct. Biol. 2, 593-596 (1992).

The term “patient” refers to a human patient.

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal nonhuman animal, such as a transgenic mouse, having a genome comprising a human heavy chain transgene and a light chain transgene, fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further elsewhere herein), (b) antibodies isolated from a host cell transformed to express the antibody, such as from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies may be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The terms “transgenic, non-human animal” refers to a non-human animal having a genome comprising one or more human heavy and/or light chain transgenes or transchromosomes (either integrated or non-integrated into the animal's natural genomic DNA) and which is capable of expressing fully human antibodies. For example, a transgenic mouse can have a human light chain transgene and either a human heavy chain transgene or human heavy chain transchromosome, such that the mouse produces human anti-CD20 antibodies when immunized with CD20 antigen and/or cells expressing CD20. The human heavy chain transgene may be integrated into the chromosomal DNA of the mouse, as is the case for transgenic mice, for instance the HuMAb-Mouse®, such as HCo7 or HCo12 mice, or the human heavy chain transgene may be maintained extrachromosomally, as is the case for the transchromosomal KM-Mouse® as described in WO 02/43478. Such transgenic and transchromosomal mice (collectively referred to herein as “transgenic mice”) are capable of producing multiple isotypes of human monoclonal antibodies to a given antigen (such as IgG, IgA, IgM, IgD and/or IgE) by undergoing V-D-J recombination and isotype switching. Transgenic, nonhuman animals can also be used for production of antibodies against a specific antigen by introducing genes encoding such specific antibody, for example by operatively linking the genes to a gene which is expressed in the milk of the animal.

For amino acid (polypeptide) sequences, the term “identity” or “homology” indicates the degree of identity between two amino acid sequences when optimally aligned and compared with appropriate insertions or deletions. The percent identity between two sequences is a function of the number of identical positions shared by the sequences (i.e., % identity=number of identical positions/total number of positions times 100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two polypeptide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two amino acid sequences can also be determined using the algorithm of E. Meyers and W. Miller (Comput. Appl. Biosci., 4:11-17 (1988)) which has been incorporated into the ALIGN program (version 2.0), using a PAM 120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

By way of example, a polypeptide sequence may be identical to a polypeptide reference sequence as described herein (for example SEQ ID NO: 1) that is be 100% identical, or it may include up to a certain integer number of amino acid alterations as compared to the reference sequence such that the % identity is less than 100%, such as at least 50, 60, 70, 75, 80, 85, 90, 95, 98, or 99% identical. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by the polypeptide reference sequence as described herein (for example SEQ ID NO: 1) by the numerical percent of the respective percent identity (divided by 100) and then subtracting that product from said total number of amino acids in the polypeptide reference sequence as described herein (for example SEQ ID NO: 1), or:

nₐ≦xₐ−(xₐ·y),

wherein nₐ is the number of amino acid alterations, xₐ is the total number of amino acids in the polypeptide sequence encoded by SEQ ID NO: 1, and y is, 0.50 for 50%, 0.60 for 60%, 0.70 for 70%, 0.75 for 75%, 0.80 for 80%, 0.85 for 85%, 0.90 for 90%, 0.95 for 95%, 0.98 for 98%, 0.99 for 99%, or 1.00 for 100%, · is the symbol for the multiplication operator, and wherein any non-integer product of xₐ and y is rounded down to the nearest integer prior to subtracting it from xₐ.

In one embodiment of the invention, the composition is formulated in accordance with routine procedures as a pharmaceutical composition adapted for intravenous administration to human beings. Typically, compositions for intravenous administration are solutions in sterile isotonic aqueous buffer. Where suitable, the composition may also include a solubilizing agent and a local anesthetic, such as lignocaine, to ease pain at the site of the injection. Generally, the ingredients are supplied either separately or mixed together in unit dosage form, for example, as a dry lyophilized powder, or water-free concentrate, in a hermetically sealed container, such as an ampoule or sachette, indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An anti-CD20 antibody of the present invention may be administered via any suitable route, such as an oral, nasal, inhalable, intrabronchial, intraalveolar, topical (including buccal, transdermal and sublingual), rectal, vaginal and/or parenteral route. In one embodiment, a pharmaceutical composition of the present invention is administered subcutaneously (SC), optionally intramuscularly, typically by injection.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In one embodiment, a formulation for an anti-CD 20 antibody (including ofatumuamb) can be formulated according to a formulation disclosed in WO2009/009407.

In one embodiment an anti-CD20 antibody pharmaceutical composition is administered in crystalline form by subcutaneous injection, cf. Yang et al., PNAS USA 100(12), 6934-6939 (2003).

Pharmaceutically acceptable carriers include any and all suitable solvents, dispersion media, coatings, antibacterial and antifungal agents, isotonicity agents, antioxidants and absorption delaying agents, and the like that are physiologically compatible with a compound of the present invention.

Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

Proper fluidity may be maintained, for example, by the use of coating materials, such as lecithin, by the maintenance of the required particle size in the case of dispersions, and by the use of surfactants.

Pharmaceutical compositions containing an anti-CD20 antibody may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical compositions containing an anti-CD20 antibody may also comprise isotonicity agents, such as sugars, polyalcohols such as mannitol, sorbitol, glycerol or sodium chloride in the compositions.

Pharmaceutically acceptable diluents include saline and aqueous buffer solutions.

The pharmaceutical compositions containing an anti-CD20 antibody may also contain one or more adjuvants appropriate for the chosen route of administration, such as preservatives, wetting agents, emulsifying agents, dispersing agents, preservatives or buffers, which may enhance the shelf life or effectiveness of the pharmaceutical composition. An anti-CD20 antibody the present invention may for instance be admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol. Other examples of adjuvants are QS21, GM-CSF, SRL-172, histamine dihydrochloride, thymocartin, Tio-TEPA, monophosphoryl-lipid A/microbacteria compositions, alum, incomplete Freund's adjuvant, montanide ISA, ribi adjuvant system, TiterMax adjuvant, syntex adjuvant formulations, immune-stimulating complexes (ISCOMs), gerbu adjuvant, CpG oligodeoxynucleotides, lipopolysaccharide, and polyinosinic:polycytidylic acid.

Prevention of presence of microorganisms may be ensured both by sterilization procedures and by the inclusion of various antibacterial and antifungal agents, for example, paraben, chlorobutanol, phenol, sorbic acid, and the like. In addition, prolonged absorption of the injectable pharmaceutical form may be brought about by the inclusion of agents which delay absorption, such as aluminum monostearate and gelatin.

The pharmaceutical compositions containing an anti-CD20 antibody may be in a variety of suitable forms. Such forms include, for example, liquid, semi-solid and solid dosage forms, such as liquid solutions (e.g., injectable and infusible solutions), dispersions or suspensions, emulsions, microemulsions, gels, creams, granules, powders, tablets, pills, powders, liposomes, dendrimers and other nanoparticles (see for instance Baek et al., Methods Enzymol. 362, 240-9 (2003), Nigavekar et al., Pharm Res. 21(3), 476-83 (2004), microparticles, and suppositories.

The optimal form depends on the mode of administration chosen and the nature of the composition. Formulations may include, for instance, powders, pastes, ointments, jellies, waxes, oils, lipids, lipid (cationic or anionic) containing vesicles, DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing may be appropriate in treatments and therapies in accordance with the present invention, provided that the anti-CD20 antibody in the pharmaceutical composition is not inactivated by the formulation and the formulation is physiologically compatible and tolerable with the route of administration. See also for instance Powell et al., “Compendium of excipients for parenteral formulations” PDA J Pharm Sci Technol. 2, 238-311 (1998) and the citations therein for additional information related to excipients and carriers well known to pharmaceutical chemists.

An anti-CD20 antibody may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers, such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art. See e.g., Sustained and Controlled Release Drug Delivery Systems, J. R. Robinson, ed., Marcel Dekker, Inc., New York, 1978.

To administer the pharmaceutical compositions containing an anti-CD20 antibody by certain routes of administration according to the invention, it may be necessary to coat the anti-CD20 antibody with, or co-administer the antibody with, a material to prevent its inactivation. For example, the anti-CD20 antibody may be administered to a subject in an appropriate carrier, for example, liposomes, or a diluent. Liposomes include water-in-oil-in-water CGF emulsions as well as conventional liposomes (Strejan et al., J. Neuroimmunol. 7, 27 (1984)).

Depending on the route of administration, an anti-CD20 antibody may be coated in a material to protect the antibody from the action of acids and other natural conditions that may inactivate the compound. For example, the anti-CD20 antibody may be administered to a subject in an appropriate carrier, for example, liposomes. Liposomes include water-in-oil-in-water CGF emulsions as well as conventional liposomes (Strejan et al., J. Neuroimmunol. 7, 27 (1984)).

Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated. Supplementary active compounds may also be incorporated into the compositions.

Pharmaceutical compositions for injection must typically be sterile and stable under the conditions of manufacture and storage. The composition may be formulated as a solution, microemulsion, liposome, or other ordered structure suitable to high drug concentration. The carrier may be a aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating, such as lecithin, by the maintenance of the required particle size in the case of dispersion and by the use of surfactants. In many cases, it will be preferable to include isotonic agents, for example, sugars, polyalcohols, such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration.

Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

The present invention may be embodied in other specific forms, without departing from the spirit or essential attributes thereof, and, accordingly, reference should be made to the appended claims, rather than to the foregoing specification or following examples, as indicating the scope of the invention.

The method according to the invention may also comprise the step of administering additional pharmaceutically active agents with the anti-CD20 antibody. Suitable additional pharmaceutically active agents include, but are not limited to, firategrast, fingolimod, natalizumab, methotrexate, interferon-gamma, cyclophosphamide, corticosteroids such as prednisone and prednisolone, non-steroidal anti-inflammatory drugs (NSAIDs), such as paracetamol, and anti-histamines, such as diphenhydramine.

It is postulated that drugs such as firategrast and natalizumab may sequester B-cells by blocking migration or egress of such cells from germinal centres, and thus, combining such therapies with anti-CD20 antibody therapy may result in an enhancement of the cell-killing effect of the anti-CD20 antibody. Accordingly, in one aspect, the invention provides a method of treating, arresting or preventing a disease responsive to treatment with an anti-CD20 antibody in a patient suffering therefrom, comprising administering to the patient an anti-CD20 antibody and one or more of firategrast, fingolimod and natalizumab.

Prior use of anti-CD20 antibodies in autoimmune disease settings such as rheumatoid arthritis have typically involved pre-medication with corticosteroids. In an embodiment, patients treated with the dosage regimens of the present invention do not receive pre-medication with corticosteroids.

As used herein, the term, “carrier”, refers to a diluent, adjuvant, excipient, or vehicle with which the therapeutic is administered.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from at least one of its coexisting cellular materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

As used herein, the term, “pharmaceutical”, includes veterinary applications of the invention. The term, “therapeutically effective amount”, refers to that amount of therapeutic agent, which is useful for alleviating a selected condition.

As used herein, the term, “pharmaceutically acceptable”, means approved by a regulatory agency of the Federal or a state government or listed in the U.S. Pharmacopeia or other generally recognized pharmacopeia for use in animals, and more particularly in humans.

“Week one”, as used herein, may refer to, the 8^{th} day of treatment (wherein the initial dose is administered to the patient on day 1 of treatment), “week 12” as used herein may refer to the 85^{th} day of treatment, and “every four weeks” as used herein may refer to administration of anti-CD20 antibody at approximately every 28 days. It will be understood that the exact timing of the administration—i.e. the exact day of delivery—may not be critical. Thus, for instance, delivery may be within +/−7 days of the stated day, +/−5 days of the stated day, +/−3 days of the stated day, +/−2 days of the stated days or +/−1 day of the stated day. For example, in one embodiment, delivery could be on day 1, day 8 (+/−3 days), day 85 (+/−3 days).

### Example 1

### Pharmacokinetics and Pharmacodynamics of Subcutaneously Administered Ofatumumab in RA Patients on Stable Methotexate

Background: Ofatumumab (OFA), a fully human monoclonal antibody (MAb), targets a novel epitope on the CD20 molecule distinct from rituximab (RTX), a chimeric anti-CD20 MAb. RTX studies have utilized high intravenous (IV) doses (two 1000 mg doses 14 days apart) leading to very rapid B cell lysis resulting in infusion reactions despite the use of IV corticosteroid (CS) premedication. This phase I/II study investigates the use of a low dose subcutaneous (SC) formulation of OFA, administered without CS, potentially providing more controlled B cell depletion.

Objectives: The primary objective was to investigate the safety and tolerability of a single SC dose of OFA in rheumatoid arthritis (RA) patients on background methotrexate (MTX). Secondary objectives included investigating the minimum dose to achieve target peripheral B-cell depletion, the pharmacodynamic dose-response curve and B-cell repletion profile.

Methods: This was a multicentre, single-blind, placebo (PBO)-controlled, dose escalation/de-escalation study in RA patients on stable MTX. Eligible patients were randomised into five cohorts to receive a single OFA dose (0.3, 3, 30, 60, or 100 mg) or PBO after pre-medication with oral paracetamol and oral antihistamine. Target peripheral B-cell depletion by group was defined as ≧95% from baseline, or to below the lower limit of quantification (LLQ), as measured by median change at Week 4 and/or the median value across weeks 2-4. Repletion was defined as return of peripheral B-cells to either ≧baseline or ≧lower limit of normal. Start of repletion was defined as B-cell count <95% depletion or ≧10 cells/mm³ on 2 consecutive occasions. Data was analysed when the last patient reached Day 169.

Results: Thirty-five patients were recruited as follows: 0.3 mg, n=4; 3 mg, n=6; 30 mg, n=8; 60 mg, n=6; 100 mg, n=3; PBO, n=8. After a single 30, 60 or 100 mg SC dose, OFA was absorbed with median tmax values ranging from 4.02 to 4.49 days; the elimination mean t1/2 values ranged from 5.84 to 7.23 days. The OFA levels after the 0.3 and 3 mg dosing were <LLQ. An increasing level of B-cell depletion from the 0.3 mg up to the 30 mg group was observed. Target depletion was achieved for the 30, 60 and 100 mg OFA groups. Seventeen patients, 1 in the 3 mg, 7 in the 30 mg, and all in the 60 mg and 100 mg groups met depletion criteria. Four of these 17 reached repletion criterion by Day 169, the earliest at Day 113. Start of repletion was achieved by 14 patients and began as early as Day 43. Overall, the incidence of adverse events (AEs) in the combined OFA groups was 89% (24/27) and 63% (5/8) with PBO. Headache, nausea and upper respiratory infection were the most commonly reported AEs (≧5 subjects in the combined OFA groups). AEs considered to be post-injection systemic reactions (PISRs) occurred in 48% (13/27) and 25% (2/8) of patients in the combined OFA and PBO groups, respectively. Most were graded as mild with only 3 severe AEs reported in 2 patients: pyrexia (60 mg) and nausea and headache (100 mg). Only 3 patients were recruited to the 100 mg cohort due to tolerability concerns (PISRs). There were no injection site reactions or positive human anti-human antibodies.

Conclusions: In this study of RA patients on stable MTX doses, SC OFA doses of 30, 60 or 100 mg resulted in profound and sustained peripheral B-cell depletion. Single doses up to 60 mg were tolerated and may provide a method of achieving B-cell depletion without additional CS premedication.

Sequence Listing

| | | |
|---|---|---|
| SEQ ID NO: 1 | 2F2 V_{H} | EVQLVESGGGLVQPGRSLRL |
| | | SCAASGFTFNDYAMHWVRQA |
| | | PGKGLEWVSTISWNSGSIGY |
| | | ADSVKGRFTISRDNAKKSLY |
| | | LQMNSLRAEDTALYYCAKDI |
| | | QYGNYYYGMDVWGQGTTVTV |
| | | SS |
| SEQ ID NO: 2 | 2F2 V_{L} | EIVLTQSPATLSLSPGERAT |
| | | LSCRASQSVSSYLAWYQQKP |
| | | GQAPRLLIYDASNRATGIPA |
| | | RFSGSGSGTDFTLTISSLEP |
| | | EDFAVYYCQQRSNWPITFGQ |
| | | GTRLEIK |
| SEQ ID NO: 3 | 2F2 V_{H} CDR1 | DYAMH |
| SEQ ID NO: 4 | 2F2 V_{H} CDR2 | TISWNSGSIGYADSVKG |
| SEQ ID NO: 5 | 2F2 V_{H} CDR3 | DIQYGNYYYGMDV |
| SEQ ID NO: 6 | 2F2 V_{L} CDR1 | RASQSVSSYLA |
| SEQ ID NO: 7 | 2F2 V_{L} CDR2 | DASNRAT |
| SEQ ID NO: 8 | 2F2 V_{L} CDR3 | QQRSNWPIT |
| SEQ ID NO: 9 | 11B8 V_{H} CDR3 | DYYGAGSFYDGLYGMDV |
| SEQ ID NO: 10 | 2F2 V_{H} CDR1- | DYAMHWVRQAPGKGLEWVST |
| | CDR3 | ISWNSGSIGYADSVKGRFTI |
| | | SRDNAKKSLYLQMNSLRAED |
| | | TALYYCAKDIQYGNYYYGMD |
| | | V |

### REFERENCES CITED HEREIN

Alter A, Duddy M, Hebert S et al., Determinants of Human B Cell Migration Across Brain Endothelial Cells, J Immunol 2003 170(9):4497-4505
Barnett, M H & Prineas, J W, Relapsing and remitting multiple sclerosis: Pathology of the newly forming lesion, Ann Neurol 2004 55:4:458-468
Bar-Or. A. et al., Abnormal B-cell cytokine responses a trigger of T-cell mediated disease in MS?, Ann Neurol 2010:67:4:452, 461
Bleeker W K, Munk M E, Mackus W J M et al., Estimation of dose requirements for sustained in vivo activity of a therapeutic human anti-CD20 antibody, Br J Hœmatology 2007:140:303-312.
Boven, L A et al, Myelin-laden macrophages are anti-inflammatory, consistent with foam cells in multiple sclerosis, Brain 2006 129:2:517-526
Compston, A., Complexity and heterogeneity in demyelinating disease, Brain 2007 130:5:1178-1180
Cook S D, in Handbook of multiple sclerosis, ed. Cook S D 2006, 4^{th} edn., Taylor & Francis:New York
Hauser S L, Waubant E, Arnold D L, et al. B-cell Depletion with Rituximab in Relapsing-Remitting Multiple Sclerosis. N Engl J Med 2008; 358:676-88.
Healy B C, Ikle D, Macklin E A, Cutter G. Optimal design and analysis of phase I/II clinical trials in multiple sclerosis with gadolinium-enhanced lesions as the endpoint. Mult Scler 2010; 16:840-847.
Haas. J. et al., Prevalence of Newly Generated Naive Regulatory T Cells (Treg) Is Critical for Treg Suppressive Function and Determines Treg Dysfunction in Multiple Sclerosis, J. Immunol 2007 179:2:1322-1330
Haas, J. et al., Glatiramer acetate improves regulatory T-cell function by expansion of naïve CD4+CD25+FOXP3+CD31+ T-cells in patients with multiple sclerosis, J. Immunol 2009:216:113-117
Kuenz B, Lutterotti A, Ehling R et al., Cerebrospinal Fluid B Cells Correlate with Early Brain Inflammation in Multiple Sclerosis, PLoS one 2008:3(7):e2559
Macallan, D C, Wallace D, Zhang Y et al., Rapid Turnover of Effector Memory CD4+ T Cells in Healthy Humans, J Exp Med 2004:200(2):255-260
Minagar. A & Alexander, J S, Blood brain barrier disruption in multiple sclerosis, Multiple Sclerosis 2003:9:540-549
Kap, Y. S. et al. Late B Cell Depletion with a Human Anti-Human CD20 IgG1 Monoclonal Antibody Halts the Development of Experimental Autoimmune Encephalomyelitis in Marmosets. J Immunol 2010:185:7:3390-4003
Petereit H F and Rubbert-Roth A, Rituximab levels in cerebrospinal fluid of patients with neurological autoimmune disorders, Multiple Sclerosis 2009:15(2): 189-192
Polman C H, Reingold S C, Barkhof F. Ethics of placebo-controlled clinical trials in multiple sclerosis; A reassessment. Neurology 2008; 70:1134-1140.
Sorensen P S, Drulovic J, Havrdova E. Magnetic Resonance Imaging (MRI) Efficacy of Ofatumumab in Relapsing-Remitting Multiple Sclerosis (RRMS)—24-Week Results of a Phase II Study 2010; Abstract in Multiple Sclerosis (In Press).
Sormani M P, Bruzzi P, Rovaris M et al., Modelling new enhancing MRI lesion counts in multiple sclerosis, Multiple Sclerosis 2001:7:298-304
Tourtellotte W W, 1975, What is multiple sclerosis?Laboratory criteria for diagnosis, Multiple Sclerosis Research, ed. Davison A N, Humphrey J H, Liversedge A L et al., p. 9, HMSO:London

1. A method of treating, arresting or preventing multiple sclerosis comprising in a human patient comprising administering an anti-CD20 antibody at:
(a) initial 3 mg dose followed by 30 mg at week one, and 30 mg at week 12; or
(b) intial 3 mg dose followed by 60 mg at week one, and 60 mg at week 12; or
(c) initial 3 mg dose followed by 60 mg at every four weeks for 24 weeks; or
(d) initial 3 mg dose followed by 30 mg at every four weeks for 24 weeks; or
(e) initial 3 mg dose, followed by 10 mg at every twelve weeks for 24 weeks; or
(f) 30 mg at week one, and 30 mg at week 12; or
(g) 60 mg at week one, and 60 mg at week 12; or
(h) 60 mg at every four weeks for 24 weeks; or
(i) 30 mg at every four weeks for 24 weeks; or
(j) 3 mg at week one, and 3 mg at week 12; or
(k) 10 mg at week one, and at every twelve weeks for 24 weeks.

2. The method of claim 1 in which administration is subcutaneous.

3. The method of claim 1 in which multiple sclerosis is relapse-remitting multiple sclerosis.

4. The method of claim 1 in which multiple sclerosis is primary progressive multiple sclerosis.

5. The method of claim 1 in which multiple sclerosis is secondary progressive multiple sclerosis.

6. A method of treating, arresting or preventing patient spino-optical sclerosis or neuromyelitis optica in a human patient comprising administering an anti-CD20 antibody at
(a) initial 3 mg dose followed by 30 mg at week one, and 30 mg at week 12; or
(b) intial 3 mg dose followed by 60 mg at week one, and 60 mg at week 12; or
(c) initial 3 mg dose followed by 60 mg at every four weeks for 24 weeks; or
(d) initial 3 mg dose followed by 30 mg at every four weeks for 24 weeks; or
(e) initial 3 mg dose, followed by 10 mg at every twelve weeks for 24 weeks; or
(f) 30 mg at week one, and 30 mg at week 12; or
(g) 60 mg at week one, and 60 mg at week 12; or
(h) 60 mg at every four weeks for 24 weeks; or
(i) 30 mg at every four weeks for 24 weeks; or
(j) 3 mg at week one, and 3 mg at week 12; or
(k) 10 mg at week one, and at every twelve weeks for 24 weeks.

7. The method of claim 1 in which the anti-CD20 is ofatumuamb.

8. The method of claim 1 in which the anti-CD20 is rituximab.

9. The method of claim 1 in which the anti-CD20 is ocrelizumab.
